(19) United States
(12) Patent Application Publication
(10) Pub. No.: US 2020/0187466 A1
(43) Pub. Date: Jun. 18, 2020

(54) METHOD FOR CONSTRUCTING MOUSE MODEL WITH CONDITIONAL KNOCKOUT OF TMEM30A GENE FROM PANCREATIC BETA CELL, AND USE THEREOF

(71) Applicant: Xianjun ZHU, Chengdu (CN)

(72) Inventor: Xianjun ZHU, Chengdu (CN)

(21) Appl. No.: 16/615,975

(22) PCT Filed: May 18, 2018
(86) PCT No.: PCT/CN2018/087525

§ 371 Date: Nov. 22, 2019

(30) Foreign Application Priority Data
May 25, 2017 (CN) 201710380326.5

(51) Int. Cl. A01K 67/027; C12N 15/85; C12N 15/90; C12N 15/89

(52) U.S. Cl. CPC A01K 67/0276; C12N 15/8509; C12N 15/907; C12N 15/89; A01K 2217/077; C12N 2015/8536; A01K 2217/075; A01K 2227/105; A01K 2267/0362; C12N 2015/859

(57) ABSTRACT

Provided are a method for constructing a mouse model with conditional knockout of the Tmem30a gene from a pancreatic β cell and a use thereof. The construction method includes the steps of: constructing a homozygote mouse with conditional knockout of a Tmem30a gene, where both ends of one or more exons of the Tmem30a gene are inserted into directly arrayed loxp loci; and mating the mouse with a pancreatic β cell specific transgenic mouse Ins2-Cre, thereby obtaining the mouse model with conditional knockout of the Tmem30a gene from the pancreatic β cell. The mouse with conditional knockout of the Tmem30a gene from the pancreatic β cell shows glucose intolerance and poor insulin sensitivity, and can be used as a diabetes research model.

This application claims priority of Chinese Patent Application No. 201710380326.5, filed on May 25, 2017 and entitled “METHOD FOR CONSTRUCTING MOUSE MODEL WITH CONDITIONAL KNOCKOUT OF TMEM30A GENE FROM PANCREATIC β CELL, AND USE THEREOF”, which is herein incorporated by reference in its entirety.

### SEQUENCE LISTING

This application contains a Sequence Listing which has been submitted in ASCII format via EFS-Web and is hereby incorporated by reference herein in its entirety. The ASCII text file was created on Feb. 17, 2020, is named ZHU1—Sequence listing.txt and is 3,187 bytes in size.

### TECHNICAL FIELD

The present invention relates to the technical field of medical engineering, and in particular to a method for constructing a mouse model with conditional knockout of a Tmem30a gene from a pancreatic β cell, and a use thereof.

### BACKGROUND

The distribution of phospholipid molecules on the cell membrane of a eukaryotic cell is asymmetric. In general, phosphatidylserine (PS) and phosphatidylethanolamine (PE) are distributed in the inner membrane of a cell, and phosphatidylcholine (PC) is distributed in the outer membrane. A eukaryotic genome encodes 14 P4-type ATPase flippases to maintain such an asymmetric distribution of lipid molecules. The asymmetric distribution of PS and PE on the cell membrane is critical for important cellular physiological processes such as membrane stabilization, regulation of a blood coagulation reaction, transportation of vesicle proteins, and clearance of apoptotic cells. Mutations in ATP8B1, ATP8A2 and ATP11C genes have led to several human diseases, revealing the importance of P-type ATPases. The ATP8B1 causes progressive familial intrahepatic cholestasis type I and recurrent intrahepatic cholestasis. The ATP8A2 mutation causes cerebellar ataxia, mental retardation, and balance deficiency syndrome. Deletion of the ATP11C causes B cell development deficiency, anemia, and intrahepatic cholestasis.

The P4-type ATPase requires binding with a protein Tmem30 for proper folding and transporting. The Tmem30 has a similar function as that of the β subunit of a Na—K ATPase, and participates in the catalytic reaction process of the P4-type ATPase. The eukaryotic genome encodes three Tmem30 proteins, and thus it is required that one Tmem30 protein binds multiple P4-type ATP enzymes. Tmem30a is widely expressed in multiple tissues, and is also specifically expressed in photoreceptor cells of the retina. On human chromosomes, Tmem30a is located on the short arm of the chromosome 6, and consists of 7 exons. Its transcript has a size of 2 kb, and the protein encoded by it has a size of 44 kD and is generally expressed in various tissues.

By sequence analysis, the Tmem30a is highly conserved in eukaryotes, contains two membrane-anchored regions, and has a glycosylation site. The in vivo function of the Tmem30a is still unclear, and research on it is still at a preliminary stage. It is necessary to systematically study its function by constructing animal and cell models.

The increasing incidence of diabetes mellitus (DM) has become a public health problem that seriously endangers human health. DM can cause multiple organ complications in patients, which not only seriously affects the life quality of a patient, but also can lead to disability and death. Currently, the pathogenesis of DM is still not understood and unclear. A suitable animal model of DM is very important for elucidating the pathogenesis of DM and its complications.

### SUMMARY

In view of this, the present invention constructs a mouse model with specific knockout of Tmem30a from a pancreatic β cell by utilizing a pancreatic meCre transgenic mouse, so as to study its function in a pancreatic island.

Accordingly, an aspect of the present invention is intended to provide a method for constructing a mouse model of a Tmem30a gene with a pancreatic e transgenic mouse. Another aspect of the present invention is intended to provide a mouse model with conditional knockout of a Tmem30a gene from a pancreatic β cell for DM study.

In a first aspect, the present invention provides a method for constructing a mouse model with knockout of a Tmem30a gene with a pancreatic e transgenic mouse, including the steps of:

1) cloning a 5′ arm homologous to a mouse Tmem30a gene, an expression cassette containing a reporter gene LacZ, an expression cassette having a NEO resistance gene, a 3rd exon having directly arrayed loxp loci at both ends thereof, and a 3′-terminal arm into a BAC vector for replacement of the 3rd exon of the Tmem30a gene to be knocked out;

2) replacing the 3rd exon in the Tmem30a gene by using a DNA homologous recombination technology, to obtain a mouse embryo stem cell with conditional knockout of the Tmem30a gene;

3) obtaining a chimeric mouse containing the cell with the knockout of the Tmem30a gene by preparation by using the embryonic stem cells obtained in step 2);

4) mating and breeding the chimera mouse obtained in step 3) with a wild-type mouse, and screening out a heterozygote mouse with the knockout of the Tmem30a gene in the offspring;

5) mating and breeding the heterozygote mouse animal obtained in step 4) with a transgenic mouse FLPer to obtain a heterozygote mouse with conditional knockout of the Tmem30a gene;

6) intermating and breeding the heterozygote mouse with conditional knockout of the Tmem30a gene obtained in step 5) to obtain a homozygote mouse with conditional knockout of the Tmem30a gene; and

7) mating the homozygote mouse with conditional knockout of the Tmem30a gene obtained in step 6) with a pancreatic β cell specific transgenic mouse Ins2-Cre, to obtain a mouse Tmem30a loxp/loxp, Ins2-Cre with conditional knockout of the Tmem30a gene from the pancreatic β cell.

Furthermore, in some embodiments of the present invention, in step 2), a mouse embryonic stem cell is transfected with a targeting construct Tmem30a tm1a(KOMP)Wtsi with the knockout of Tmem30a, to obtain an embryonic stem cell containing the targeting sequence; and the targeting sequence has the following features:

the 5′-terminal long arm is 4201 bp; and the 3′-terminal long arm is 5123 bp. Placed in the second intron of the Tmem30a are a En2 splicing accepting site, an IRES followed by a LacZ gene expression sequence, a ployA sequence;

the Loxp locus is followed by a human βactin promoter and a neomycin coding sequence, for drug screening;

additionally, there are two FRT sites at both ends to delete a reporter gene using a FLP tool mouse.

The 3rd exon has directly arrayed Loxp sequences at both ends thereof, so as to use Cre to delete the 3rd exon and establish a tissue-specific knockout mouse model (see FIG. 1).

Furthermore, in some embodiments of the present invention, in step 3), the specific preparation method is: the embryonic stem cell obtained in the single step 2) is microinjected into the embryo sac of a mouse, and transplanted into the uterus of a pseudopregnant animal, so as to deliver a chimeric animal containing Temm30a mutant cells.

Furthermore, in some embodiments of the present invention, in step 4), after the chimeric animal integrated into a germline is mated with a wild-type animal C57BL/6J, the resultant animal of the first filial generation is screened by a long-distance PCR to obtain heterozygote individuals with knockout of the Tmem30a gene; mating the heterozygote with knockout of the Tmem30a gene with a mouse with knockin of a FLPer gene to delete a reporter gene between two FRT sites, so as to obtain a mouse heterozygote individual Tmem30a loxp/+ with conditional knockout containing two Loxp loci.

Furthermore, in some embodiments of the present invention, the primer pair used for amplifying the 5′-terminal long arm in the long-distance PCR includes GF3 and LAR3, and the base sequence of the GF3 primer is shown in SEQ ID No: 1, and the base sequence of the LAR3 primer is shown in SEQ ID No: 2.

Furthermore, in some embodiments of the present invention, the primer pair used for amplifying the 3′-terminal long arm in the long-distance PCR includes RAF5 and GR3, and the base sequence of the RAF5 primer is shown in SEQ ID No: 3, and the base sequence of the GR3 primer is shown in SEQ ID No: 4.

The present inventors have found through tests that, the homozygote mouse with systematic knockout of the Tmem30a gene is died within 9.5-12.5 days of the embryonic period, and the heterozygote mouse Tmem30a KO/+ with the knockout of the Tmem30a gene is successfully delivered.

According to partial or all of the steps of the present invention, the present invention can provide a heterozygote mouse Tmem30a loxp/+ with conditional knockout of the Tmem30a gene and a homozygote mouse Tmem30a loxp/loxp, as well as a mouse Tmem30a loxp/loxp, Ins2-Cre with conditional knockout of a Tmem30a gene from a pancreatic β cell.

In another aspect, the present invention provides a use of above-described mouse model with conditional knockout of a Tmem30a gene from a pancreatic β cell, where the mouse model with conditional knockout of a Tmem30a gene from a pancreatic β cell is used as a diabetes research model.

The inventors have found that, the above-described mouse model with conditional knockout of a Tmem30a gene from a pancreatic β cell as provided by the present invention exhibits glucose intolerance and poor insulin sensitivity, and thus can be used as a diabetes research model.

In another aspect, the present invention provides a use of the above-described mouse model with conditional knockout of a Tmem30a gene from a pancreatic β cell in screening for a medicament for preventing or treating DM.

Furthermore, in some embodiments of the present invention, a candidate drug is administrated to the mouse model with conditional knockout of a Tmem30a gene from a pancreatic β cell, where a blood glucose concentration level X1 of the mouse model with conditional knockout of a Tmem30a gene from a pancreatic β cell before the administration of the candidate drug is detected, and a blood glucose concentration level X2 of the mouse model with conditional knockout of a Tmem30a gene from a pancreatic β cell after the administration of the candidate drug is detected, and if X2 is significantly lower than X1, then it indicates that the candidate drug can be used as a medicament for treating or preventing DM.

### BRIEF DESCRIPTION OF THE DRAWING

FIG. 1 is a schematic diagram of a vector targeting for a Tmem30a mutation;

FIG. 2 is a restriction map of a Tmem30a targeting construct, where the Tmem30a targeting construct only has 1 AscSI restriction site, and becomes a linear plasmid after enzyme digestion.

FIG. 3 shows a test result of screening the transfected mouse embryonic stem cells by amplifying the 5′-terminal long arm through the long-distance PCR in Embodiment 1, where the used primer pair is GF3 and LAR3, and the amplified product is 5.8 Kb.

FIG. 4 shows a test result of screening the transfected mouse embryonic stem cells by amplifying the 3′-terminal long arm through the long-distance PCR in Embodiment 1, where the used primer pair is RAF5 and GR3, and the amplified product is 6.6 Kb.

FIG. 5 shows a test result of identifying positive mice of the first filial generation through the long-distance PCR in Embodiment 2, where the primer pair GF3 and LAR3 is used for amplifying the 5′-terminal long arm, and the amplified product is 5.8 Kb; and the primer pair RAF5 and GR3 is used for amplifying the 3′-terminal long arm, and the amplified product is 6.6 Kb; where: 204-1 is a positive heterozygote, and 204-2 is a wild-type control.

FIG. 6 is a schematic diagram showing the constructing of the Tmem30a conditional knockout model in Embodiment 3.

FIG. 7 shows the genotype identification result of the Tmem30a knockout heterozygote in Embodiment 3, where: (a) is the PCR identification result of the loxp locus upstream of the 3rd exon, and the amplified fragment is 220 bp; (b) is the PCR identification result of the loxp locus upstream of the human βactin promoter, and the amplified fragment is 214 bp; and (c) is the PCR identification result of the loxp locus downstream of the 3rd exon, the mutant amplified fragment is 214 bp, and the wild-type amplified fragment is 179 bp.

FIG. 8 shows an test result of PCR identification of a Tmem30a conditional knockout mouse for PCR identification of the loxp locus downstream of the 3rd exon, where: the wild-type amplified fragment is 179 bp (lanes 1, 4); the homozygote (loxp/loxp) amplified fragment is 214 bp (lane 3); and there are two heterozygote (loxp/+) amplified fragments: 214 bp and 179 bp (lane 2).

FIG. 9 shows building a pancreatic β cell specific knockout animal model (referred to as Ins2-Tmem30a KO for short) by mating with a pancreatic heterozygote (loxCre (Ins2-Cre). Two matings are required to obtain the pancreatic β cell specific knockout mouse Ins2Tmem30a KO.

FIG. 10 shows that the PCR identification of the pancreatic β cell specific knockout mouse Ins2 Tmem30a KO requires the use of the primer pair Tmem-Loxp-F2: attccccttcaagatagctac (SEQ ID No: 9); where

the Tmem-Loxp-R2: aatgatcaactgtaattcccc (SEQ ID No: 10) is subjected to a PCR reaction for PCR identification of the Loxp locus downstream of the 3rd exon. The wild-type amplified fragment is 179 bp (WT); the homozygote (loxp/loxp) amplified fragment is 214 bp; and there are two heterozygote (loxp/+) amplified fragments: 214 bp and 179 bp; additionally, the Ins2-Cre transgene is genotyped and the primer pair as used is: Cre-F, 5′-atttgcctgcattaccggtc-3′ (SEQ ID No: 11); and Cre-R, 5′-atcaacgttttcttttcgg-3′ (SEQ ID No: 12). The amplified PCR product fragment is 350 bp, and the wild-type has no amplified fragment.

FIG. 11 shows the body weight detection results of the mouse with knockout of the Tmem30a from the pancreatic β cell and a wild-type mouse. It shows a weight gain in the mouse Ins2 Tmema30a KO.

FIG. 12 shows glucose intolerance in the mouse Ins2 Tmema30a KO.

FIG. 13 shows that the mouse Ins2 Tmema30a KO is poorly sensitive to insulin.

FIG. 14 shows an immunofluorescence detection result and a Western blotting result of the mouse with knockout of the Tmem30a from the pancreatic β cell in Embodiment 7; in the figure: A is the result of immunofluorescence staining, and B is the result of Western blotting.

FIG. 15 shows the results of staining and area detection of subcutaneous fat cells of the mouse with knockout of the Tmem30a from the pancreatic β cell in Embodiment 9; in the figure: the left side is the subcutaneous fat western section staining, and the right side is the statistical result of the subcutaneous fat cell area detection.

FIG. 16 shows a detection result of blood glucose concentration in a 3-month-old mouse with knockout of the Tmem30a from the pancreatic β cell after fasting and glucose injection in Embodiment 10; in the figure: A—the intravenous glucose of the knockout mouse is higher than a control after fasted for 12 hours; B—the glucose tolerance test shows a decrease in glucose tolerance in the knockout mouse; and C—the area under the curve (AUC) of time-blood glucose for different month-aged mice.

FIG. 17 shows a detection result of blood glucose concentration in a 4-month-old mouse with knockout of the Tmem30a from the pancreatic β cell after fasting and glucose injection in Embodiment 11.

FIG. 18 shows a result of H&E staining of a mouse pancreatic paraffin section in Embodiment 13.

FIG. 19 shows the observation results of sizes of a core vesicle, a Golgi apparatus, an endoplasmic reticulum, and a mitochondria in a β cell of the mouse with knockout of the Tmem30a from the pancreatic β cell by a transmission electron microscopy (TEM) in Embodiment 14.

FIG. 20 shows liver fat accumulation in the mouse Ins2 Tmema30a KO.

### DETAILED DESCRIPTION

The present invention will be described in detail below in connection with specific embodiments. It will be understood that, the following embodiments are only used for illustrating the present invention, rather than limiting the scope of the present invention. The test methods and techniques for which the specific conditions are not noted in the following embodiments are generally carried out according to the conventional conditions in the art to which the present invention pertains or according to the conditions recommended by the manufacturer.

The features and performances of the present disclosure are further described in detail below in conjunction with the embodiments.

### Embodiment 1 Acquisition of Tmem30a Heterozygote Mouse

1) A targeting construct Tmem30a tm1a(KOMP)Wtsi (purchased from Children's Hospital Oakland Research Institute, USA) was linearized, and then was used to transfect a mouse embryonic stem cell 129Sv through electric shock, the embryonic stem cells were expanded and cultured, 500 clones were screened out, so as to give two embryonic stem cell strains G6 and A11 containing correct targeting sequences.

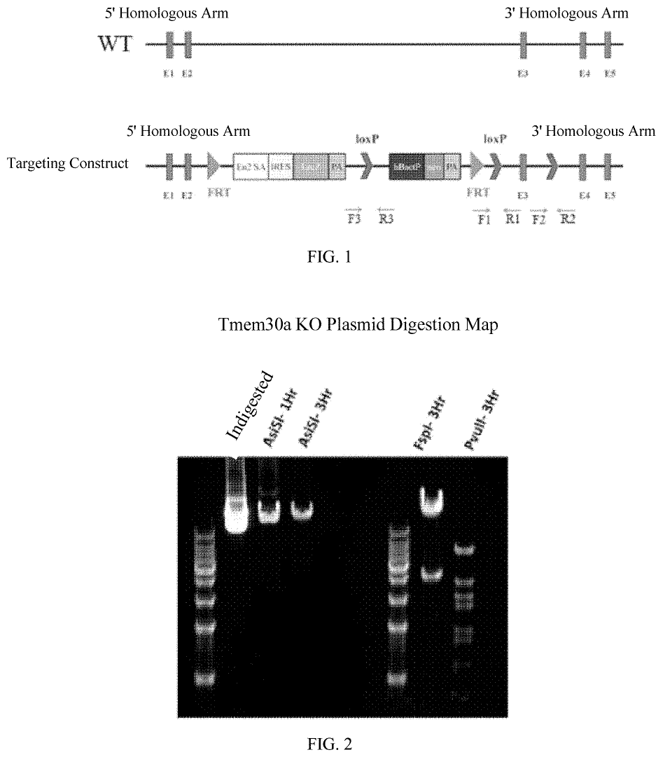

The Tmem30a targeting construct Tmem30a tm1a(KOMP)Wtsi had a structure shown in FIG. 1, the 5′-terminal long arm was 4201 bp, and the 3′-terminal long arm was 5123 bp; placed in the second intron was a En2 splicing accepting (SA) site, an IRES followed by a LacZ gene coding sequence, a ployA sequence (PA); the loxp locus was followed by a human βactin promoter and a neomycin coding sequence (Neo), for drug screening; additionally, there were two FRT sites at both ends to delete a reporter gene using a FLP tool mouse; and the 3rd exon (E3) had directly arrayed Loxp sequences at both ends thereof, so as to use Cre to delete the 3rd exon and establish a tissue-specific gene knockout mouse model.

This Embodiment 1 is illustrated by taking the 3rd exon as a specific example, and the present invention included, but was not limited to, adding directly arrayed Loxp loci at both ends of the 3rd exon to construct a mouse with conditional knockout of a gene. In the present invention, directly arrayed Loxp loci also could be added to both ends of the other exons such as the 1st, the 2nd, the 4th, the 5th, the 6th or the 7th exon to construct a conditional gene knockout mouse.

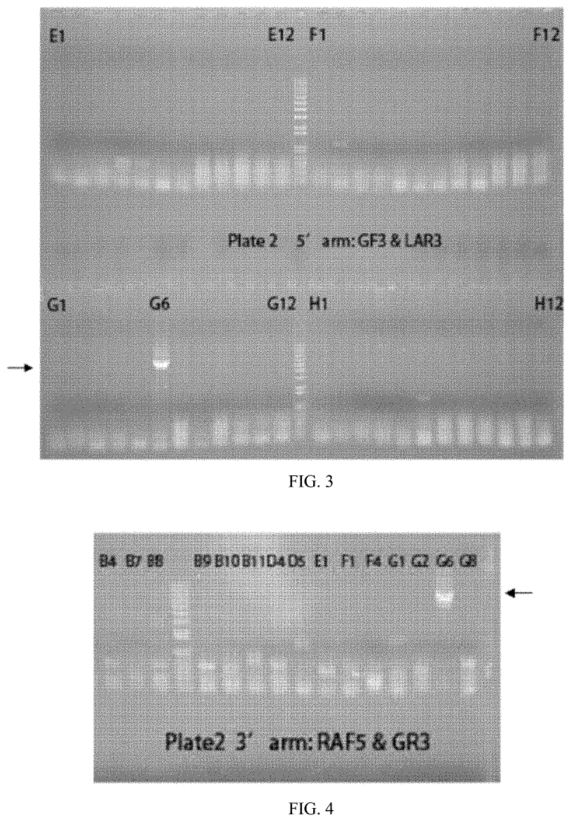

The targeting construct shown in FIG. 1 was linearized by digestion with an AsiSI endonuclease for 2 hours, as shown in FIG. 2.

2) The clone G6 screened out in the amplification step 1) was digested by trypsinization into individual cells, and injected into the blastocyst of a C57BL/6J mouse by micro-blastocyst injection, and the embryos were transplanted into the uterus of a pseudopregnant mouse to obtain a chimeric male mouse integrated with Tmem30a mutant cells. The male chimeric mouse was mated with a wild-type female mouse, and the resultant mice were screened by PCR for heterozygote mice with the knockout of Tmem30a (referred to as Tmem30a KO for short), and the heterozygote mice were named Tmem30aTm1Xzhu.

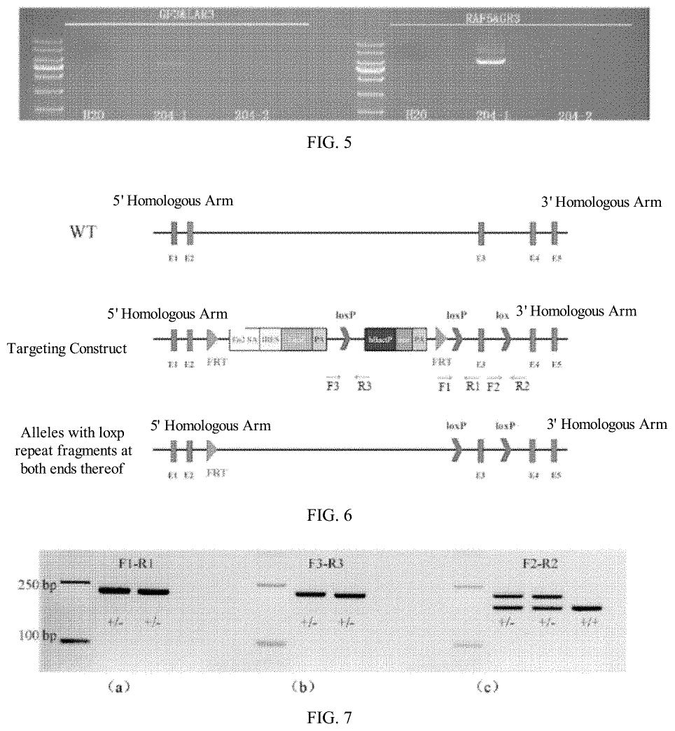

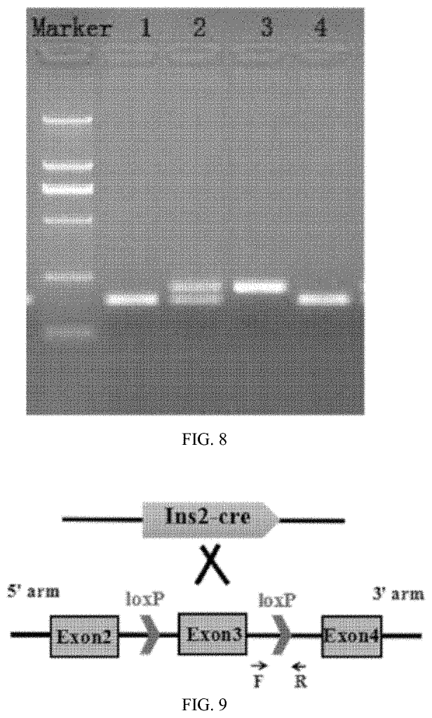

FIGS. 3 and 4 were the results of screening transfected mouse embryonic stem cells through the long distance PCR. The primer pair GF3 and LAR3 was used for amplifying the 5′-terminal long arm, and the amplified product was 5.8 Kb (FIG. 3). The primer pair RAF5 and GR3 was used for amplifying the 3′-terminal long arm, and the amplified product was 6.6 Kb (FIG. 4). Only G6 in the second 96-well plate contains the correct 5′-terminal and 3′-terminal long arms. The sequences of respective primers were as follows:

GF3:
(SEQ ID No: 1)
5′-GAGGAAGCGGAAGTGTAAGTTACCAAG-3′
LAR3:
(SEQ ID No: 2)
5′-CACAACGGGTTCTTCTGTTAGTCC-3′;
RAF5:
(SEQ ID No: 3)
5′-CACACCTCCCCCTGAACCTGAAAC-3′;
GR3:
(SEQ ID No: 4)
5′-GTGTGAAGTCAACGTCATTATCGGAGAATC-3′.

### Embodiment 2 the Homozygote Mouse with Knockout of the Tmem30a was Died within 9.5-12.5 Days of the Embryonic Period

Mice with a C57BL/6/129Sv heterozygote background were selected as the test mice.

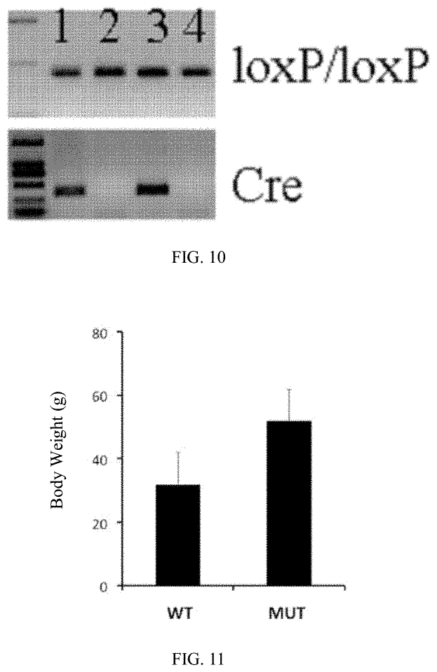

The Tmem30a KO heterozygote mouse obtained in Embodiment 1 was mated with a C57BL/6J mouse (purchased from Jackson Laboratory, USA), and the obtained Tmem30a KO heterozygote mouse with the C57BL/6/129Sv heterozygote background could be born normally and conformed to Mendelian's law. There was no significant difference between the Tmem30a KO heterozygote mice and wild-type mice. We tested the progenies produced by intermating of the Tmem30a KO heterozygote mice by PCR and the like methods. The results were shown in FIG. 5, and it was found that no surviving Tmem30a KO homozygote mice were born. We then conducted statistics analysis on their progenies, with the wild-type and heterozygote accounting for 1/3 and 2/3, respectively (Table 1). This result was consistent with the Mendelian inheritance law in which homozygote embryos were lethal.

TABLE 1
Statistical analysis of the progenies of intermating
of Tmem30a KO heterozygote mice

| | Heterozygote | Wild Type | Homozygote |
|---|---|---|---|
| The number of progenies on | 42 | 22 | 0 |
| the first day of birth | | | |
| The percentage (%) of the | 65.6 | 34.4 | 0 |
| number of progenies | | | |
| on the first day of birth | | | |
| accounting for the total | | | |
| number of born mice | | | |
| The estimated percentage | 50 | 25 | 25 |
| (%) under the | | | |
| Mendelian inheritance law | | | |

To determine the exact embryo death time of the Temm30a KO homozygote mouse, we isolated embryos of 9.5-12.5 days. Combined with genotypic detection means such as PCR, and by embryo morphology observation, it was found that there was no Tmem30a KO homozygote embryo in embryos of 12.5 days; in embryos of 9.5 and 10.5 days, Tmem30a KO homozygotes had a retarded development with individuals being smaller than wild-type and heterozygote mice, and the individual differences are more pronounced as function of days.

### Embodiment 3 Construction of Tmem30a Conditional Knockout Mice

The lethal Tmem30a KO homozygotes had affected the in-depth study of its function. In order to study the in vivo function of Tmem30a in various tissues in detail, it was necessary to establish a Tmem30a conditional knockout mouse.

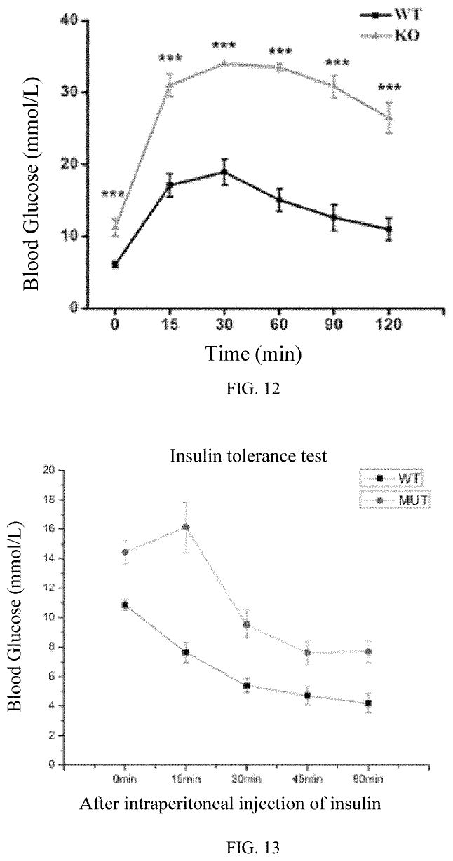

The Tmem30a KO heterozygote was mated with FLP deleter (introduced from Jackson Laboratory, USA, with the strain name of B6129S4-Gt(ROSA)26Sortm1(FLP1)Dym/RainJ, also known as FLPer) mouse, and in the genome of the progeny as born, the En2-IRES-LacZ-hACT-Neo sequence between the two FRTs would be deleted, leaving only the loxp loci at both ends of the 3rd exon (see FIG. 6). This animal model was a Tmem30a conditional knockout model named Tmem30aTm11Xzhu, which was referred to as Tmem30a loxp for short. Mating of Tmem30a loxp/+ heterozygotes with C7BL/6J could expand the population size of heterozygotes. Mating of Tmem30a loxp/+ heterozygotes could give homozygotes Tmem30a loxp/loxp.

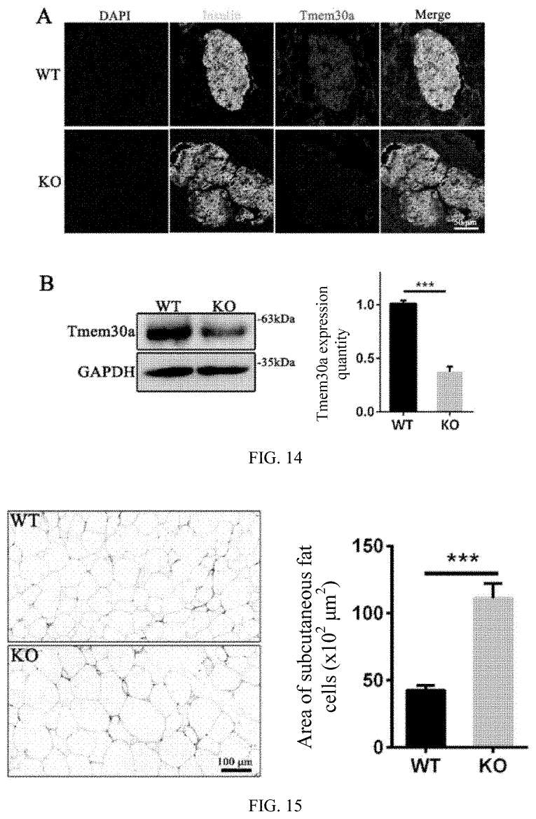

FIG. 7 showed genotype identification results of the Tmem30a KO heterozygote, where: (a) the Tmem30a knockout heterozygote was detected using a PCR reaction for PCR identification of the loxp locus upstream of the 3rd exon, where it required the use of the following primer pair:

Tmem-Loxp-F1:
(SEQ ID No: 5)
5′-gtcgagaagttcctattccga-3′;
Tmem-Loxp-R1:
(SEQ ID No: 6)
5′-tcttcaaatgtttgcccta-3′;

the amplified fragment was 220 bp.

(b) a PCR reaction was used for PCR identification of the loxp locus upstream of the human βactin promoter, where it required the use of the following primer pair:

Tmem-Loxp-F3:
(SEQ ID No: 7)
5′-CACTGCATTCTAGTTGTGGTT-3′;
Tmem-Loxp-R3:
(SEQ ID No: 8)
5′-GGACATCTCTTGGGCACTGA-3′;

the amplified fragment was 214 bp.

(c) a PCR reaction was used for PCR identification of the loxp locus downstream of the 3rd exon, where it required the use of the following primer pair:

Tmem-Loxp-F2:
(SEQ ID No: 9)
5′-attccccttcaagatagctac-3′;
Tmem-Loxp-R2:
(SEQ ID No: 10)
5′-aatgatcaactgtaattcccc-3′;

The mutant amplified fragment was 214 bp (Mutant), and the wild-type amplified fragment was 179 bp (WT).

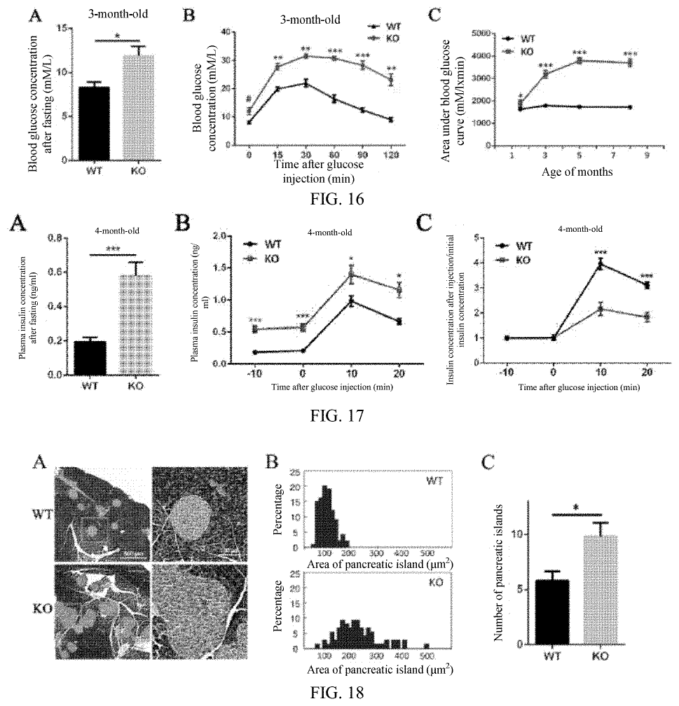

FIG. 8 showed the PCR identification result of the Tmem30a conditional knockout mouse, where it required the use of the following primer pair:

Tmem-Loxp-F2:
(SEQ ID No: 9)
5′-attccccttcaagatagctac-3′;
Tmem-Loxp-R2:
(SEQ ID No: 10)
5′-aatgatcaactgtaattcccc-3′;

The loxp locus downstream of the 3rd exon was identified through the PCR reaction, where the wild-type amplified fragments in the 1st and 4th lanes were each 179 bp (WT); there were two heterozygote (flox/+) amplified fragments in the 2nd lane: 214 bp and 179 bp; and the homozygote (loxp/loxp) amplified fragment in the 3rd lane was 214 bp.

### Embodiment 4 Construction of Mouse with Knockout of the Tmem30a from the Pancreatic β Cell

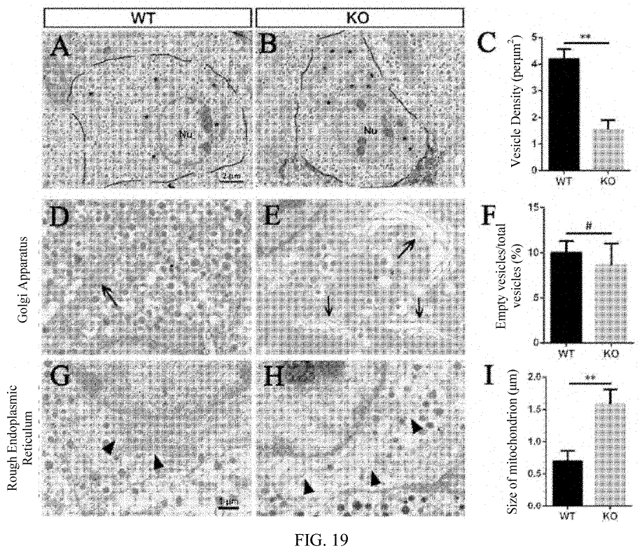

The Tmem30a loxp/loxp homozygote was mated with a pancreatic β cell specific transgenic Cre (B6Cg-Tg(Ins2-cre) 25 Mgn/J, referred to as Ins2-Cre for short) mouse (FIG. 9), and thus a Tmem30a loxp/+,Ins2-Cre heterozygote could be obtained. The heterozygote was then mated with the Tmem30a loxp/loxp homozygote, such that a pancreatic β cell specific knockout Tmem30a loxp/loxp,Ins2-Cre mouse could be obtained, which was referred to as Ins2-Tmema30a KO mouse for short.

The Loxp locus downstream of the 3rd exon was subjected to PCR identification by PCR reaction using the primer pair Tmem-Loxp-F2 and Tmem-Loxp-R2. As shown in FIGS. 10, 1, 2, 3, and 4 were four animals from one nest, and the amplified fragment was 214 bp, all of the mice were homozygotes (loxp/loxp).

Additionally, the Ins2-Cre transgene was genotyped and the primer pair as used was:

Cre-F:
(SEQ ID No: 11)
5′-atttgcctgcattaccggtc-3′;
Cre-R:
(SEQ ID No: 12)
5′-atcaacgttttcttttcgg-3′.

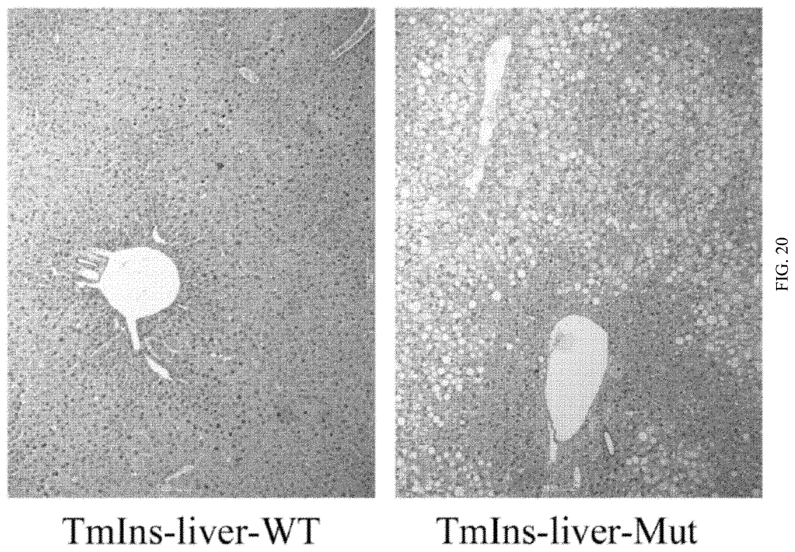

As shown in FIG. 10, the amplified PCR product fragment of the Cre transgene was 350 bp (lanes 1 and 3), and the wild-type had no amplified fragment (lanes 2 and 4).

### Embodiment 5 the Mouse with Knockout of the Tmem30a from the Pancreatic β Cell had an Abnormal Body Weight and Abnormal Blood Glucose Metabolism

As compared with the control group (expressed as WT, with the genotype of Tmem30a loxp/loxp), the Tmem30a pancreatic β cell knockout mouse knockout animal (expressed as MUT, with the genotype of Tmem30a loxp/loxp,Ins2-Cre) homozygote animal had an average body weight of 51 grams at the age of 7 months, with an increase of 40% as compared with the control (FIG. 11). Glucose tolerance test (GTT) demonstrated that, the MUT animal had glucose intolerance, with the blood glucose being rapidly increased to 33 mmol/L after glucose injection and the venous blood glucose being significantly higher than that of the control group (FIG. 12). The insulin tolerance test (ITT) demonstrated that, the MUT animal was not sensitive to insulin, with the blood glucose not being decreased rapidly as that in the control group after insulin injection, and the venous blood glucose being significantly higher than that of the control group (FIG. 13).

### Embodiment 6 Tmem30a was Specifically Knocked Out from the Pancreatic β Cell

The immunofluorescence staining showed that the Tmem30a expression was deleted from the pancreatic β cell of the mouse with knockout of the Tmem30a from the pancreatic β cell (FIG. 14-A), and additionally, the Western blotting showed that the quantity of Tmem30a expression of the mouse with knockout of the Tmem30a from the pancreatic β cell was significantly decreased (FIG. 14-B). The above results indicated that Tmem30a was specifically knocked out from the pancreatic β cell of the mouse with knockout of the Tmem30a from the pancreatic β cell.

The immunofluorescence staining method was as follows: the mouse pancreas was fixed and sectioned for immunohistochemical analysis, and stained for Tmem30a and insulin separately. The specific steps were as follows:

1. a pancreatic tissue was fixed, dehydrated, embedded and then sectioned;

2. the sections were baked in a 37° C. oven for 45 min;

3. the sections were blocked with a blocking solution (formulated in donkey serum) for 2 h;

4. the sections were stained with primary antibodies (antibodies against Tmem30a and insulin) at 4° C. overnight;

5. the sections were washed with PBS for 3 times, with 10 min for each time;

6. the sections were stained with secondary antibodies at room temperature for 2 h;

7. the sections were washed with PBS for 3 times, with 10 min for each time;

8. the sections were mounted with a mounting medium, and was observed by a confocal microscopy.

The results showed that the Tmem30a expression was deleted from the pancreatic β cell of the knockout mouse (KO).

B. The mouse pancreas was removed for immunoblotting, and the specific steps were as follows:

1. the pancreatic endocrine glands were removed and homogenized, and lysed on ice with the addition of appropriate amount of RIPA lysate for 20 min;

2. the lysed solution was ultrasonically disrupted until the lysed solution was clear and free of precipitation;

3. the lysed solution was added with a Loading buffer and boiled in a water bath for 5 min;

4. the solution was centrifuged at 12000 g for 5 min, and the supernatant was taken;

5. electrophoretic separation: 15 μl-2 μl of the sample was loaded onto the SDS-PAGE gel (10 cm×10 cm) for electrophoresis; and

6. membrane transfer: the gel was immersed in a transfer buffer for 10 min for equilibration, and 6 pieces of membranes and filter papers were clipped according to the size of the gel and were placed into the transfer buffer for 10 min for equilibration. If a PVDF membrane was used, then it should be immersed in pure methanol for 3-5 seconds for saturation, and the transfer sandwich should be assembled, where after each layer was placed in place, the bubbles were removed by using a test tube. The gel was placed on a cathode surface (the black side). A transfer trough was placed into an ice bath, the sandwich was placed therein (facing the black side to the black side), a transfer buffer was added, and an electrode was inserted at 100V for 1 h (with the current of about 0.3 A). After the membrane transfer was finished, the power was turned off, and the hybridization membrane was taken out;

7. the hybridization membrane was blocked with 8% skim milk at room temperature for 2 h;

8. the hybridization membrane was incubated overnight together with a primary antibody on a 4° C. shaker;

9. the hybridization membrane was rinsed with PBS for 3 times, with 10 min for each time;

10. the hybridization membrane was incubated together with a secondary antibody at room temperature on a shaker for 2 h;

11. the hybridization membrane was rinsed with PBS for 3 times, with 10 min for each time;

12. an exposure solution was formulated, and then exposure was conducted on an exposure machine.

The results showed that the expression level of the KO mouse Tmem30a in the pancreatic cell was significantly reduced.

### Embodiment 7

The subcutaneous fat cell area detection result showed that the subcutaneous fat cell of the mouse with knockout of the Tmem30a from the pancreatic β cell was enlarged with the subcutaneous fat cell area being significantly larger than that of a wild-type mouse (FIG. 15), indicating fat accumulation in the mouse with knockout of the Tmem30a from the pancreatic β cell.

The detection method was as follows:

The mouse abdominal fat was fixed, embedded in paraffin, sectioned and then stained with HE, and the specific steps were as follows:

1. the tissue was fixed, dehydrated, dipped in wax, embedded, and then sectioned;

2. then the sections were dewaxed and hydrated, and stained with a hematoxylin solution or a Masson dye solution for about 5-15 min;

3. excess dye was washed off with distilled water;

4. the sections were added with a diluted alcohol hydrochloride solution for color separation, and microscopic examination was conducted at the same time as the color separation until the nucleus was reddish purple and the cytoplasm was colorless;

5. after the color separation, the sections were alkalized with tap water for turning back to blue;

6. the sections were then dyed with a Eosin dye solution, and subjected to color separation of Eosin with 95% alcohol, until the cytoplasm, connective tissue, etc. were pink;

7. the stained sections were immersed into ethanol solutions of concentrations ascending from 70% to 100% for dehydration; and

8. the sections were immersed into a xylene transparent agent twice (each for several minutes), taken out, dropped with a neutral gum, and sealed with a cover slip.

As a result, it was found that the subcutaneous fat cells of the knockout mouse were enlarged, indicating fat accumulation.

### Embodiment 8

The mouse with knockout of the Tmem30a from the pancreatic β cell showed an early phenotype of type 2 diabetes, and the venous blood glucose of the mouse with knockout of the Tmem30a from the pancreatic β cell was higher than that of the wild-type after 12 hours of fasting (FIG. 16-A). The glucose tolerance test showed a decrease in the glucose tolerance of the mouse with knockout of the Tmem30a from the pancreatic β cell (FIG. 16-B). The result of the area under the curve of time-blood glucose for different month-aged mice showed that the AUC under the blood glucose curve of the mouse with knockout of the Tmem30a from the pancreatic β cell was higher than that of the wild-type (FIG. 16-C).

After the mice were injected with glucose, blood of them was taken for glucose tolerance tests, and the specific steps were as follows:

1. the mice were fasted for 12 h;

2. the mice were intraperitoneally injected with a 15% glucose solution (0.5-2 g/kg), and the blood glucose concentrations of the mice were measured at 0, 15, 30, 60, 90, and 120 min after the injection, respectively; and

3. a concentration curve was drawn statistically.

The results showed that the venous blood glucose of the knockout mouse was higher than that of the control after fasting for 12 hours (FIG. 16-A), and the glucose tolerance test showed a decrease in the glucose tolerance of the knockout mouse (FIGS. 16-B, and 16-C).

### Embodiment 9

After fasting, the plasma insulin concentration of the KO mouse was higher than that of the wild-type mouse, and the KO mouse exhibited hyperinsulinemia (FIG. 17-A). The insulin secretion test showed that at 10 min and 20 min of glucose injection, the plasma insulin concentration of the KO mouse was higher than that of the wild-type mouse (FIG. 17-B). After the glucose injection, the ratio of the insulin concentration after the injection/the initial insulin concentration of the KO mouse was lower than that of the wild-type mouse (FIG. 17-C). The above results indicated that the insulin secretion of the KO mouse was relatively insufficient.

After the mice were injected with glucose, blood of them were taken for insulin secretion tests, and the specific steps were as follows:

1. the mice were fasted for 12 h;

2. the blood was taken from the mice at the orbite 10 min before the glucose injection;

3. the mice were intraperitoneally injected with a 15% glucose solution (0.5-2 g/kg), and the blood was taken from the mice at the orbite at 0, 10, and 20 min of the injection, respectively;

4. the collected blood sample was centrifuged at 3000 rpm, and the supernatant was taken;

5. plasma insulin concentration measurement was conducted according to the steps of a ELISA insulin measurement kit; and

6. a concentration curve was drawn statistically.

The results showed that the plasma insulin concentration of the knockout mouse was higher than that of the control (Panel A), but it indicated that the insulin secretion of the knockout mouse was relatively insufficient (Panels B and C).

### Embodiment 10

H&E staining of mouse pancreatic paraffin sections showed hyperplasia of the pancreatic islet in the knockout mouse (FIG. 18).

The mouse pancreas was fixed, embedded in paraffin, sectioned and then stained with HE, and the specific steps were as follows:

1. the tissue was fixed, dehydrated, dipped in wax, embedded, and then sectioned;

2. then the sections were dewaxed and hydrated, and stained with a hematoxylin solution or a Masson dye solution for about 5-15 min;

3. excess dye was washed off with distilled water;

4. the sections were added with a diluted alcohol hydrochloride solution for color separation, and microscopic examination was conducted at the same time as the color separation until the nucleus was reddish purple and the cytoplasm was colorless;

5. after the color separation, the sections were alkalized with tap water for turning back to blue;

6. the sections were then dyed with a Eosin dye solution, and subjected to color separation of Eosin with 95% alcohol, until the cytoplasm, connective tissue, etc. were pink;

7. the stained sections were immersed into ethanol solutions of concentrations ascending from 70% to 100% for dehydration; and

8. the sections were immersed into a xylene transparent agent twice (each for several minutes), taken out, dropped with a neutral gum, and sealed with a cover slip.

It was found that, the pancreatic island of the knockout mouse had an enlarged volume and an increased number, indicating hyperplasia of the pancreatic island (FIG. 18).

### Embodiment 11

The transmission electron microscopy (TEM) results showed that the number of dense core vesicles in the pancreatic β cell of the KO mouse was significantly decreased as compared with the control, and there were subcellular phenotypes such as expansion of Golgi apparatus and endoplasmic reticulum as well as mitochondrial enlargement, indicating that the Tmem30a deficiency leaded to endoplasmic reticulum stress (FIG. 19).

The pancreatic endocrine glandstron of the mouse was taken and fixed, and then observed under an electronmicroscope, and the specific steps were as follows:

1. sampling: the samples were taken accurately and rapidly, and the size of the tissue block was less than 1 cubic millimeter;

2. fixing:

the sample was fixed in a formulation of 2.5% glutaraldehyde and a phosphate buffer for 2 hours or longer,

the sample was rinsed with a 0.1M phosphate rinse buffer (15 min, three times),

the sample was fixed in a 1% osmic acid fixative (2-3 hours), and

the sample was rinsed with a 0.1M phosphate rinse buffer (15 min, three times);

3. dehydration:

50% ethanol for 15-20 min,

70% ethanol for 15-20 min,

90% ethanol for 15-20 min,

90% ethanol and 90% acetone (1:1) for 15-20 min, and

90% acetone for 15-20 min,

the above operations were carried out in a 4° C. refrigerator,

dehydration was conducted in 100% acetone at room temperature for three times, each time for 15-20 min;

4. embedding:

pure acetone+embedding solution (2:1), at room temperature for 3-4 hours,

pure acetone+embedding solution (1:2), at room temperature overnight,

pure embedding solution, at 37° C. for 2-3 hours;

5. curing:

a 37° C. oven, overnight,

a 45° C. oven, 12 hours, and

a 6° C. oven, 48 hours;

6. slicing in a ultra-thin slicer at 70 nm;

7. double staining in 3% uranyl acetate-lead citrate; and

8. observing under a transmission electron microscope JEOL JEM-1230 (8 KV), and photographing.

The results showed that, the transmission electron microscopy (TEM) results showed that the number of dense core vesicles in the pancreatic β cell of the knockout mouse was significantly decreased as compared with the control, and there were subcellular phenotypes such as expansion of Golgi apparatus and endoplasmic reticulum as well as mitochondrial enlargement, indicating that the Tmem30a deficiency leaded to endoplasmic reticulum stress.

### Embodiment 12 the Liver of the Mouse with Knockout of the Tmem30a from the Pancreatic β Cell had Fat Accumulation and an Abnormal Structure

We observed the H&E staining of fixed liver sections of the wild-type and the mouse with knockout of the Tmem30a from the pancreatic β cell at the age of 9 months (FIG. 20).

The liver was fixed, embedded in paraffin, sectioned and then stained with Masson, and the specific steps were as follows:

1. the tissue was fixed, dehydrated, dipped in wax, embedded, and then sectioned;

2. then the sections were dewaxed and hydrated, and stained with a hematoxylin solution or a Masson dye solution for about 5-15 min;

3. excess dye was washed off with distilled water;

4. the sections were added with a diluted alcohol hydrochloride solution for color separation, and microscopic examination was conducted at the same time as the color separation until the nucleus was reddish purple and the cytoplasm was colorless;

5. after the color separation, the sections were alkalized with tap water for turning back to blue;

6. the sections were then dyed with a Eosin dye solution, and subjected to color separation of Eosin with 95% alcohol, until the cytoplasm, connective tissue, etc. were pink;

7. the stained sections were immersed into ethanol solutions of concentrations ascending from 70% to 100% for dehydration; and

8. the sections were immersed into a xylene transparent agent twice (each for several minutes), taken out, dropped with a neutral gum, and sealed with a cover slip.

It was found that the liver of the mouse with knockout of the Tmem30a from the pancreatic β cell had fat accumulation and contained a large amount of oil droplet particles (FIG. 20).

The above results fully demonstrate that the mouse model constructed by the method for constructing a mouse model with conditional knockout of a Tmem30a gene from a pancreatic β cell as provided by the present invention has typical DM characteristics and is suitable for use in DM study, which provides the basis for further understanding the mechanism of DM and screening for DM drugs.

The series of detailed description listed above are only specific illustration of feasible embodiments of the present invention, rather than limiting the claimed scope of the present invention. All equivalent embodiments or changes made without departing from the technical spirit of the present invention should be included in the claimed scope of the present invention.

Industrial applicability: the method for constructing a mouse model with conditional knockout of a Tmem30a gene from a pancreatic β cell as disclosed in the present invention can construct a mouse model with knockout of a Tmem30a gene from a pancreatic β cell, which exhibits a typical DM model and can be used in DM study, which provides the basis for further understanding the mechanism of DM and screening for DM drugs.

### Sequence Information:

primer GF3:
SEQ ID NO: 1
5′-GAGGAAGCGGAAGTGTAAGTTACCAAG-3′,
primer LAR3:
SEQ ID NO: 2
5′-CACAACGGGTTCTTCTGTTAGTCC-3′,
primer RAF5:
SEQ ID NO: 3
5′-CACACCTCCCCCTGAACCTGAAAC-3′,
primer GR3:
SEQ ID NO: 4
5′-GTGTGAAGTCAACGTCATTATCGGAGAATC-3′,
primer Tmem-Loxp-F1:
SEQ ID NO: 5
5′-GTCGAGAAGTTCCTATTCCGA-3′,
primer Tmem-Loxp-R1:
SEQ ID NO: 6
5′-TCTTCAAATGTTTGCCCTA-3′,
primer Tmem-Loxp-F3:
SEQ ID NO: 7
5′-CACTGCATTCTAGTTGTGGTT-3′,
primer Tmem-Loxp-R3:
SEQ ID NO: 8
5′-GGACATCTCTTGGGCACTGA-3′,
primer Tmem-Loxp-F2:
SEQ ID NO: 9
5′-ATTCCCCTTCAAGATAGCTAC-3′,
primer Tmem-Loxp-R2:
SEQ ID NO: 10
5′-AATGATCAACTGTAATTCCCC-3′,
primer Cre-F:
SEQ ID NO: 11
5′-ATTTGCCTGCATTACCGGTC-3′,
primer Cre-R:
SEQ ID NO: 12
5′-ATCAACGTTTTCTTTTCGG-3′,

1. A method for constructing a mouse model with conditional knockout of a Tmem30a gene from a pancreatic β cell, comprising the steps of:
1) cloning a 5′ arm homologous to a mouse Tmem30a gene, an expression cassette containing a reporter gene LacZ, an expression cassette having a NEO resistance gene, a 3rd exon having directly arrayed loxp loci at both ends thereof, and a 3′-terminal arm into a BAC vector for replacement of the 3rd exon of the Tmem30a gene to be knocked out;
2) replacing the 3rd exon in the Tmem30a gene by using a DNA homologous recombination technology, to obtain a mouse embryo stem cell with conditional knockout of the Tmem30a gene;
3) obtaining a chimeric mouse containing the cell with the knockout of the Tmem30a gene by preparation by using the embryonic stem cells obtained in step 2);
4) mating and breeding the chimera mouse obtained in step 3) with a wild-type mouse, and screening out a heterozygote mouse with the knockout of the Tmem30a gene in the offspring;
5) mating and breeding the heterozygote mouse animal obtained in step 4) with a transgenic mouse FLPer to obtain a heterozygote mouse with conditional knockout of the Tmem30a gene;
6) intermating and breeding the heterozygote mouse with conditional knockout of the Tmem30a gene obtained in step 5) to obtain a homozygote mouse with conditional knockout of the Tmem30a gene; and
7) mating the homozygote mouse with conditional knockout of the Tmem30a gene obtained in step 6) with a pancreatic β cell specific transgenic mouse Ins2-Cre, to obtain a mouse Tmem30a loxp/loxp, Ins2-Cre with conditional knockout of the Tmem30a gene from the pancreatic β cell.

2. The method for constructing a mouse model with conditional knockout of a Tmem30a gene from a pancreatic β cell according to claim 1, wherein
in step 2), a mouse embryonic stem cell is transfected with a targeting construct Tmem30a tm1a(KOMP)Wtsi with the knockout of Tmem30a, to obtain an embryonic stem cell containing the targeting sequence; and the targeting sequence has the following features:
the 5′-terminal long arm is 4201 bp; and the 3′-terminal long arm is 5123 bp; placed in the second intron of the Tmem30a are a En2 splicing accepting site, an IRES followed by a LacZ gene expression sequence, a ployA sequence;
the Loxp locus is followed by a human βactin promoter and a neomycin coding sequence, for drug screening;
additionally, there are two FRT sites at both ends to delete a reporter gene using a FLP tool mouse; and
the 3rd exon has directly arrayed Loxp sequences at both ends thereof, so as to use Cre to delete the 3rd exon and establish a tissue-specific knockout mouse model.

3. The method for constructing a mouse model with conditional knockout of a Tmem30a gene from a pancreatic β cell according to claim 1, wherein in
step 3), the specific preparation method is: the embryonic stem cell obtained in the single step 2) is microinjected into the embryo sac of a mouse, and transplanted into the uterus of a pseudopregnant animal, so as to deliver a chimeric animal containing Temm30a mutant cells.

4. The method for constructing a mouse model with conditional knockout of a Tmem30a gene from a pancreatic β cell according to claim 1, wherein
in step 4), after the chimeric animal integrated into a germline is mated with a wild-type animal C57BL/6J, the resultant animal of the first filial generation is screened by a long-distance PCR to obtain heterozygote individuals with knockout of the Tmem30a gene; mating the heterozygote with knockout of the Tmem30a gene with a mouse with knockin of a FLPer gene to delete a reporter gene between two FRT sites, so as to obtain a mouse heterozygote individual Tmem30a loxp/+ with conditional knockout containing two Loxp loci.

5. The method for constructing a mouse model with conditional knockout of a Tmem30a gene from a pancreatic β cell according to claim 4, wherein the primer pair used for amplifying the 5′-terminal long arm in the long-distance PCR comprises GF3 and LAR3, and the base sequence of the GF3 primer is shown in SEQ ID No: 1, and the base sequence of the LAR3 primer is shown in SEQ ID No: 2.

6. The method for constructing a mouse model with conditional knockout of a Tmem30a gene from a pancreatic β cell according to claim 5, wherein the primer pair used for amplifying the 3′-terminal long arm in the long-distance PCR comprises RAF5 and GR3, and the base sequence of the RAF5 primer is shown in SEQ ID No: 3, and the base sequence of the GR3 primer is shown in SEQ ID No: 4.

7. A use of a mouse model with conditional knockout of a Tmem30a gene from a pancreatic β cell, wherein the mouse model with conditional knockout of a Tmem30a gene from a pancreatic β cell as constructed by the construction method of claim 1 is used as a diabetes research model.

8. A mouse model with conditional knockout of a Tmem30a gene from a pancreatic β cell, wherein it is a mouse model Tmem30a loxp/loxp, Ins2-Cre with conditional knockout of the Tmem30a gene from the pancreatic β cell as constructed by the construction method of claim 1.

9. A heterozygote mouse model with conditional knockout of a Tmem30a gene, wherein it is a heterozygote mouse Tmem30a loxp/+ with conditional knockout of the Tmem30a gene as constructed by steps 1)-5) in the construction method of claim 1.

10. A homozygote mouse model with conditional knockout of a Tmem30a gene, wherein it is a homozygote mouse Tmem30a loxp/loxp with conditional knockout of the Tmem30a gene as constructed by steps 1)-6) in the construction method of claim 1.

11. A mouse model with conditional knockout of a Tmem30a gene from a pancreatic β cell, wherein any one or more of the 1st exon, the 2nd exon, the 3rd exon, the 4th exon, the 5th exon, the 6th exon, and the 7th exon is knocked out in the Tmem30a gene of this mouse model.

12. A use of the mouse model with conditional knockout of a Tmem30a gene from a pancreatic β cell according to claim 8 in screening for a medicament for preventing or treating DM.

13. The use according to claim 12, wherein a candidate drug is administrated to the mouse model with conditional knockout of a Tmem30a gene from a pancreatic β cell, in which a blood glucose concentration level X1 of the mouse model with conditional knockout of a Tmem30a gene from a pancreatic β cell before the administration of the candidate drug is detected, and a blood glucose concentration level X2 of the mouse model with conditional knockout of a Tmem30a gene from a pancreatic β cell after the administration of the candidate drug is detected, and if X2 is significantly lower than X1, then it indicates that the candidate drug can be used as a medicament for treating or preventing DM.

14. A mouse model with conditional knockout of a Tmem30a gene from a pancreatic β cell, wherein it is a mouse model Tmem30a loxp/loxp, Ins2-Cre with conditional knockout of the Tmem30a gene from the pancreatic β cell as constructed by the construction method of claim 2.

15. A mouse model with conditional knockout of a Tmem30a gene from a pancreatic β cell, wherein it is a mouse model Tmem30a loxp/loxp, Ins2-Cre with conditional knockout of the Tmem30a gene from the pancreatic β cell as constructed by the construction method of claim 3.

16. A mouse model with conditional knockout of a Tmem30a gene from a pancreatic β cell, wherein it is a mouse model Tmem30a loxp/loxp, Ins2-Cre with conditional knockout of the Tmem30a gene from the pancreatic β cell as constructed by the construction method of claim 4.

17. A heterozygote mouse model with conditional knockout of a Tmem30a gene, wherein it is a heterozygote mouse Tmem30a loxp/+ with conditional knockout of the Tmem30a gene as constructed by steps 1)-5) in the construction method of claim 2.

18. A heterozygote mouse model with conditional knockout of a Tmem30a gene, wherein it is a heterozygote mouse Tmem30a loxp/+ with conditional knockout of the Tmem30a gene as constructed by steps 1)-5) in the construction method of claim 3.

19. A homozygote mouse model with conditional knockout of a Tmem30a gene, wherein it is a homozygote mouse Tmem30a loxp/loxp with conditional knockout of the Tmem30a gene as constructed by steps 1)-6) in the construction method of claim 2.

20. A homozygote mouse model with conditional knockout of a Tmem30a gene, wherein it is a homozygote mouse Tmem30a loxp/loxp with conditional knockout of the Tmem30a gene as constructed by steps 1)-6) in the construction method of claim 3.


SEQUENCE LISTING (12 sequences)

SEQ ID NO: 1 - DNA, 27 bp, artificial sequence
  Oligonucleotide to amplify 5′ terminal long arm including GF3
gaggaagcggaagtgtaagttaccaag

SEQ ID NO: 2 - DNA, 24 bp, artificial sequence
  Oligonucleotide to amplify 5′ terminal long arm including LAR3
cacaacgggttcttctgttagtcc

SEQ ID NO: 3 - DNA, 24 bp, artificial sequence
  Oligonucleotide to amplify 3′ terminal long arm including RAF5
cacacctccccctgaacctgaaac

SEQ ID NO: 4 - DNA, 30 bp, artificial sequence
  Oligonucleotide to amplify 3′ terminal long arm including GR3
gtgtgaagtcaacgtcattatcggagaatc

SEQ ID NO: 5 - DNA, 21 bp, artificial sequence
  Oligonucleotide fw to amplify Tmem-loxp insertion (primer tmem-loxp-f1)
gtcgagaagttcctattccga

SEQ ID NO: 6 - DNA, 19 bp, artificial sequence
  Oligonucleotide rev to amplify Tmem-loxp insertion (primer tmem-loxp-r1)
tcttcaaatgtttgcccta

SEQ ID NO: 7 - DNA, 21 bp, artificial sequence
  Oligonucleotide fw to amplify Tmem-loxp insertion (primer tmem-loxp-f3)
cactgcattctagttgtggtt

SEQ ID NO: 8 - DNA, 20 bp, artificial sequence
  Oligonucleotide rev to amplify Tmem-loxp insertion (primer tmem-loxp-r3)
ggacatctcttgggcactga

SEQ ID NO: 9 - DNA, 21 bp, artificial sequence
  Oligonucleotide fw to amplify Tmem-loxp insertion (primer tmem-loxp-f2)
attccccttcaagatagctac

SEQ ID NO: 10 - DNA, 21 bp, artificial sequence
  Oligonucleotide rev to amplify Tmem-loxp insertion (primer tmem-loxp-r2)
aatgatcaactgtaattcccc

SEQ ID NO: 11 - DNA, 20 bp, artificial sequence
  Oligonucleotide fw to amplify Ins2-CRE transgene (primer cre-f)
atttgcctgcattaccggtc

SEQ ID NO: 12 - DNA, 19 bp, artificial sequence
  Oligonucleotide rev to amplify Ins2-CRE transgene (primer cre-r)
atcaacgttttcttttcgg